(19) United States
(12) Patent Application Publication
(10) Pub. No.: US 2019/0380994 A1
(43) Pub. Date: Dec. 19, 2019

(54) TREATMENT OF PANCREATIC CANCER

(71) Applicant: SynCore Biotechnology Co., Ltd., Tung Shan Shine, I-Lan (TW)

(72) Inventors: Ruey-Kuen Hsieh, Tung Shan Shine (TW); Hui-Yuan Tseng, Taipei (TW); Sih-Ting Lin, Taipei (TW)

(73) Assignee: SynCore Biotechnology Co., Ltd, Tung Shan Shine, I-Lan (TW)

(21) Appl. No.: 16/475,524

(22) PCT Filed: Jan. 4, 2018
(86) PCT No.: PCT/CN2018/071312

§ 371 Date: Jul. 2, 2019

Related U.S. Application Data
(60) Provisional application No. 62/442,636, filed on Jan. 5, 2017.

(51) Int. Cl. A61K 31/337; A61K 31/7068; A61K 9/127; A61P 35/00

(52) U.S. Cl. CPC A61K 31/337; A61P 35/00; A61K 9/127; A61K 31/7068

(57) ABSTRACT

A method of treating refractory or resistant pancreatic cancer by administering: (i) a cationic liposomal formulation comprising one or more cationic lipids and a therapeutically effective amount of paclitaxel; or (ii) a cationic liposomal formulation comprising one or more cationic lipids and a therapeutically effective amount of paclitaxel, and a therapeutically effective amount of gemcitabine.

### TECHNICAL FIELD

The present disclosure provides methods for treating pancreatic cancer after disease progression following a treatment with one or more antineoplastic agents.

### BACKGROUND

Pancreatic cancer is a highly aggressive and fatal disease with mortality rate nearly equal to incidence. As about 80% of patients are initially diagnosed with advanced disease, prognosis of pancreatic cancer is extremely poor. According to American Cancer Society, estimates of 2015 rank pancreatic cancer as the fourth leading cause of cancer-related mortality in the United States; however, it is projected to become the second leading cause of cancer death by 2030. See Cancer Res. 74: 2913-21 (2014).

For years, fluorouracil was a standard treatment for pancreatic cancer until gemcitabine showed significant improvement in median overall survival as compared with fluorouracil (5.6 v 4.4 months, P=0.002). See Burris et al., J Clin Oncol. 15(6):2403-13 (1997). Gemcitabine has been the global standard of care for the first-line treatment of advanced pancreatic cancer since 1997. Gemcitabine is administered by intravenous infusion at a dose of 1000 mg/m² over 30 minutes once weekly for up to 7 weeks, followed by a week of rest from treatment. Subsequent cycles should consist of infusions once weekly for 3 consecutive weeks out of every 4 weeks.

Numerous studies have evaluated various regimens and combinations of gemcitabine with cytotoxic or novel targeted agents over the past decade. However, few gemcitabine-based regimens demonstrated significant improvement in overall survival. In 2007, the European Commission approved erlotinib plus gemcitabine as first-line treatment for metastatic pancreatic cancer in the EU. The US FDA previously approved this combination as a first-line treatment for patients with locally-advanced, unresectable or metastatic pancreatic cancer in 2005. A phase III study showed that the treatment with erlotinib plus gemcitabine resulted in an improvement in one-year survival compared with gemcitabine alone (23% v 17%). See J Clin Oncol. 25(15):1960-66 (2007). A combination therapy of erlotinib and gemcitabine for the treatment of pancreatic cancer includes administering 100 mg erlotinib once daily in combination with 1000 mg/m² gemcitabine once weekly.

In 2006, TS-1, a drug combination of tegafur, gimeracil and oteracil, became available in Japan for the treatment of unresectable pancreatic cancer. In a phase III study, median overall survival was 8.8 months in the gemcitabine group, 9.7 months in the TS-1 group, and 10.1 months in the gemcitabine plus S-1 group; the noninferiority of TS-1 to gemcitabine was demonstrated, whereas the superiority of gemcitabine plus TS-1 was not. See J Clin Oncol. 31(13):1640-8 (2013).

In 2010, FOLFIRINOX, a drug combination consisting of leucovorin, fluorouracil, irinotecan, and oxaliplatin, emerged as a new standard therapy. FOLFIRINOX compared with gemcitabine as first-line therapy of metastatic pancreatic cancer was studied, and the median overall survival was 11.1 months in the FOLFIRINOX group as compared with 6.8 months in the gemcitabine group. FOLFIRINOX regimen for treating pancreatic cancer, for example, is consisted of a 2-hour intravenous infusion of oxaliplatin (85 mg/m²) followed by a 2-hour intravenous infusion of leucovorin (400 mg/m²) concomitantly with a 90-min intravenous infusion of irinotecan (180 mg/m²), followed by a bolus (400 mg/m²) and a 46-hour continuous infusion (2400 mg/m²) of fluorouracil. See N Engl J Med 364: 1817-25 (2011).

In 2013, albumin-bound paclitaxel was approved in the US for use in combination with gemcitabine as first-line therapy in patients with metastatic pancreatic cancer. The median overall survival was 8.5 months in the albumin-bound paclitaxel-gemcitabine group as compared with 6.7 months in the gemcitabine group. A combination therapy of albumin-bound paclitaxel and gemcitabine for the treatment of pancreatic cancer includes administering 125 mg/m² albumin-bound paclitaxel intravenously over 30-40 minutes on Days 1, 8 and 15 of each 28-day cycle, and administering 1000 mg/m² gemcitabine on Days 1, 8 and 15 of each 28-day cycle immediately after albumin-bound paclitaxel. See N Engl J Med 369:1691-703 (2013).

However, treatments for advanced pancreatic cancer over the past decade have primarily relied on only one line of therapy. This may be due to the aggressive nature of the disease and the lack of consensus on effective treatment options in second-line therapy. Therefore, there is a need in treating patients with pancreatic cancer who fail first-line treatment, such as gemcitabine alone, gemcitabine-based regimens or FOLFIRINOX. In particular, as FOLFIRINOX becomes more widely used as a first-line therapy, a second-line regimen showing efficacy and tolerability in patients who have received first-line FOLFIRINOX is required. In addition, it was reported that a low-dose continuous strategy coupled with pulsed dose may lead to the maximal delay until clinically observable resistance. See PLoS One. 2015 Nov. 4; 10(11). To choose proper cancer treatment, physicians need to consider multiple factors such as patient's age, medical history and side effect. A physician's decision may include a cancer regimen which may be more liable to drug resistance, for example, a regimen lacking a pulsed dose. For those regimens, there is particularly a need of a second-line regimen.

### SUMMARY

Provided herein are methods for treating refractory or resistant pancreatic cancer comprising administering to a subject in need thereof a cationic liposomal formulation comprising one or more cationic lipids and a therapeutically effective amount of paclitaxel. In embodiments, the cationic liposomal formulation is administered in combination with gemcitabine. The cationic liposomal formulation and gemcitabine are a combination therapy and are administered simultaneously or sequentially. In particular embodiments, the pancreatic cancer is refractory or resistant to a certain therapy, such as a first-line or second-line therapy. The methods provided herein can be used as second-line or third-line therapy.

In embodiments of the methods described herein, the subject in need thereof has been treated with a certain therapy and the pancreatic cancer has become refractory or resistance to the therapy. The subject has not been treated with any other therapies other than the therapy that the pancreatic cancer has become refractory or resistant.

In some embodiments, the subject has previously been treated with one or more antineoplastic agents comprising fluorouracil, bleomycin, bortezomib, carboplatin, cisplatin, cytarabine, docetaxel, doxorubicin, elmustin, erlotinib, etoposide, gemcitabine, idarubicin, imatinib, lomustine, methotrexate, mitomycin, mitoxantrone, oxaliplatin, paclitaxel, pemetrexed, sunitinib, topotecan, treosulfan, vemurafenib, vinblastine, vincristine, vindesine, or vinorelbine.

In some embodiments, the subject has previously been treated with fluorouracil-based combination therapy, including but not limited to a combination of oxaliplatin, leucovorin, irinotecan, and fluorouracil.

In some embodiments, the subject has previously been treated with gemcitabine-based combination therapy, including but not limited to a combination of albumin-bound paclitaxel and gemcitabine.

In some embodiments, the subject has previously been treated with an antimitotic agent selected from a group consisting of paclitaxel, docetaxel, vinblastine, vincristine, vindesine and vinorelbine. In some embodiments, the subject has previously been treated with a growth factor inhibitor selected from a group consisting of erlotinib, cetuximab, gefinitib, imatinib, panitumumab, sunitinib and vemurafenib.

Methods for treating pancreatic cancer are provided, wherein the cationic liposomal formulation comprising paclitaxel is administered on days 1, 4, 8, 11, 15, 18, 22, 25, 29, 32, 36, 39, 43 and 46 at a dose of about 1 to 60 mg/m² and gemcitabine at a dose of about 300 to 1500 mg/m² is administered on days 4, 11, 18, 25, 32, 39 and 46 of a treatment cycle of seven weeks.

Methods for treating pancreatic cancer are provided, wherein the methods comprise a first treatment cycle, which is followed by one or more subsequent treatment cycles. The first treatment cycle is a period of seven weeks, while each subsequent treatment cycle is a period of three weeks. A dosing interval between the first treatment cycle and a subsequent cycle and between two subsequent treatment cycles is one week.

In the first treatment cycle, the cationic liposomal formulation comprising paclitaxel at a dose of about 1 to 60 mg/m² is administered on days 1, 4, 8, 11, 15, 18, 22, 25, 29, 32, 36, 39, 43 and 46 and gemcitabine at a dose of about 300 to 1500 mg/m² is administered on days 4, 11, 18, 25, 32, 39 and 46. A dosing interval between the first treatment cycle and the subsequent treatment cycle is one week. In the subsequent treatment cycles, the cationic liposomal formulation comprising paclitaxel at a dose of about 1 to 60 mg/m² is administered on days 1, 4, 8, 11, 15 and 18 and gemcitabine at a dose of about 300 to 1500 mg/m² is administered on days 4, 11 and 18. A dosing interval between two subsequent treatment cycles is one week.

In some embodiments, the methods comprise administering about 1 mg/m² to about 60 mg/m² paclitaxel in the cationic liposomal formulation and about 300 mg/m² to about 1500 mg/m² gemcitabine to the subject.

In some embodiments, the method comprising administering about 11 mg/m² to about 22 mg/m² paclitaxel in the cationic liposomal formulation and about 500 mg/m² to about 1000 mg/m² gemcitabine to the subject.

### DETAILED DESCRIPTION

As used herein, the term “therapeutically effective amount” is an amount of an active agent that is sufficient to achieve the desired therapeutic result in the treated subject. The result can be reduction, amelioration, palliation, lessening, delaying, and/or alleviation of one or more of the signs, symptoms, or causes of a disease. In some embodiments, a therapeutically effective amount comprises an amount sufficient to cause a tumor to shrink or to decrease growth rate. In some embodiments, a therapeutically effective amount is an amount sufficient to prevent or delay tumor recurrence. In some embodiments, a therapeutically effective amount is an amount sufficient to inhibit, retard, slow to some extent and may stop cancer cell infiltration into peripheral organs; (iv) inhibit (i.e., slow to some extent and may stop) tumor metastasis; (v) inhibit tumor growth; (vi) prevent or delay occurrence and/or recurrence of tumor. A therapeutically effective amount can be administered in one or more administrations.

As used herein, the term “subject” is a human cancer patient. Subjects in need of a treatment (in need thereof) are subjects having pancreatic cancer. In embodiments, the subject is a human patient diagnosed with or suffering from pancreatic cancer. In particular embodiments, the subject is refractory or resistant to first-line or second-line therapy for pancreatic cancer and is in need of a second-line or third-line therapy for treatment of pancreatic cancer.

The term “pancreatic cancer” as used herein includes “locally advanced pancreatic cancer” and “metastatic pancreatic cancer.” “Locally advanced pancreatic cancer” refers to tumors that arise in pancreatic exocrine or neuroendocrine tissue, but distant metastases are absent. In contrast, “metastatic pancreatic cancer” refers to cancer spreading from the site from which it originates in the pancreas to involve another part of the body, for example, liver. In some embodiments, cancers originating from pancreatic exocrine tissue include acinar cell carcinomas, adenocarcinomas, adenosquamous carcinomas, ampullary cancers, colloid carcinomas, giant cell tumors, hepatoid carcinomas, intraductal papillary-nmeinous neoplasms, mucinous cystadenocarcinomas, pancreatoblastomas, serous cystadenocarcinomas, signet ring cell carcinomas, solid and pseudopapillary tumors, and undifferentiated carcinomas. In some embodiments, cancers originating from neuroendocrine tissue include gastrinomas, glucagonomas, insulinomas, nonfunctional islet cell tumors, somatostatinomas and vasoactive intestinal peptide-releasing tumors. In some embodiments, locally advanced pancreatic cancer is an adenocarcinoma. In further embodiments, the adenocarcinoma is ductal adenocarcinoma.

The term “growth factor inhibitor” includes, but is not limited to erlotinib, cetuximab, gefinitib, imatinib, panitumumab, sunitinib and vemurafenib.

The term “antimitotic agent” includes, but is not limited to paclitaxel, docetaxel, vinblastine, vincristine, vindesine and vinorelbine.

The term “first-line therapy” refers to the standard treatment given to a subject diagnosed with a disease. It is the initial treatment and usually accepted as the best treatment for the diagnosis.

The term “second-line therapy” refers to a treatment which is chosen after first-line treatment has failed to achieve its goal, or has side effects requiring a subject to stop using that treatment. The second-line therapy is usually used when the first-line treatment has failed, was effective previously but has since stopped working, or has side effects that are not tolerated by the subject. The term “third-line therapy” refers to treatment that is given when both the first-line therapy and the second-line therapy has failed.

As used herein a “dosage regimen” refers to a protocol used to administer a liposomal formulation or non-liposomal formulation to a subject. A dosage regimen comprises a dose and dosing interval. A dosage regimen further comprises a dosing duration. As used herein “dose” refers to an amount of an active agent given in a single administration. The interval between doses can be a desired amount of time and is referred to as the “dosing interval”. As used herein “dosing duration” refers to the period of time over which a dose is administered. As used herein, “pulsed dose” refers to a dose sharply releasing an active agent once or repeatedly. In some embodiments, a pulsed dose is a bolus dose. The unit “mg/m²” refers to an amount of an active agent per human body surface area (m²). The dose calculation refers only to the mass of the active agent (not to the lipid portion).

The term “combination therapy” as used herein includes simultaneous administration of at least two active agents to a subject or their sequential administration within a time period during which the first administered therapeutic agent is still present in the subject when the second administered therapeutic agent is administered. Combination therapy as used herein also includes administering the at least two active agents separately but at the same time.

The term “fluorouracil-based combination therapy” includes but is not limited to a combination of oxaliplatin, leucovorin, irinotecan, and fluorouracil, and a combination of leucovorin, liposomal irinotecan, and fluorouracil.

The term “gemcitabine-based combination therapy” includes but is not limited to a combination of albumin-bound paclitaxel and gemcitabine, a combination of erlotinib and gemcitabine, a combination of capecitabine and gemcitabine, and a combination of cisplatin and gemcitabine.

The term “resistant” or “refractory” refers to cancer cells that survive after treating with an active agent. Such cells may have responded to an active agent initially, but subsequently exhibited a reduction of responsiveness during treatment, or did not exhibit an adequate response to the active agent in that the cells continued to proliferate in the course of treatment with the active agent.

The term “liposome” refers to a microscopic spherical membrane-enclosed vesicle (about 50-2000 nm diameter). The term “liposome” encompasses any compartment enclosed by a lipid bilayer. Liposomes are also referred to as lipid vesicles. In order to form a liposome, the lipid molecules comprise elongated non polar (hydrophobic) portions and polar (hydrophilic) portions. The hydrophobic and hydrophilic portions of the molecule are preferably positioned at the two ends of an elongated molecular structure. When such lipids are dispersed in water they spontaneously form bilayer membranes referred to as lamellae. The lamellae are composed of two mono layer sheets of lipid molecules with their non-polar (hydrophobic) surfaces facing each other and their polar (hydrophilic) surfaces facing the aqueous medium. The membranes formed by the lipids enclose a portion of the aqueous phase in a manner similar to that of a cell membrane enclosing the contents of a cell.

Thus, the bilayer of a liposome has similarities to a cell membrane without the protein components present in a cell membrane. As used herein, the term liposome includes multilamellar liposomes, which generally have a diameter in the range of about 1 to 10 micrometers and having anywhere from two to hundreds of concentric lipid bilayers alternating with layers of an aqueous phase, and also includes unilamellar vesicles which are a single lipid layer and have a diameter in the range of about 20 to about 400 nanometers (nm), about 50 to about 300 nm, about 300 to about 400 nm, or about 100 to about 200 nm, which vesicles can be produced by subjecting multilamellar liposomes to ultrasound, by extrusion under pressure through membranes having pores of defined size, or by high pressure homogenization. The liposomes can be unilamellar vesicles, which have a single lipid bilayer, and a diameter in the range of about 25-400 nm.

The cationic liposomal formulation provided herein includes one or more cationic lipids, paclitaxel, and optionally a neutral and/or anionic lipid. As used herein, the terms “liposome”, “liposomal preparation”, and “liposomal formulation” are used synonymously throughout the present application.

The amount of cationic lipids in the cationic liposomal formulation is from about 30 mole % to about 99.9 mole %. The amount of paclitaxel in the cationic liposomal formulation is at least about 0.1 mole %. The amount of neutral and/or anionic lipid is from about 30 mole % to about 70 mole %.

In some embodiments, the amount of cationic lipids in the cationic liposomal formulation includes from about 40 mole % to about 95 mole %, about 50 mole % to about 90 mole %, about 60 mole % to about 85 mole %, about 65 mole % to about 75 mole %, or about 70 mole %.

In other embodiments, the cationic liposomal formulation includes paclitaxel in an amount of from about 0.5 mole % to about 10 mole %, about 1.0 mole % to about 8 mole %, about 2 mole % to about 6 mole %, about 5 mole %, about 2.5 mole %, or about 3.0 mole %.

Optionally, the cationic liposomal formulation includes neutral and/or anionic lipids, in an amount of from about 30 mole % to about 70 mole %, about 40 mole % to about 60 mole %, about 45 mole %, or about 55 mole %.

In embodiments, the cationic liposomal formulation has a zeta potential in the range of about 0 mV to about 100 mV or in the range of about 20 mV to about 100 mV, in about 0.05 mM KCl solution at about pH 7.5.

As used herein, the term “zeta potential” refers to a measured electrical potential of a particle, such as a liposome, measured with an instrument, such as a Zetasizer 3000 using Laser Doppler micro-electrophoresis under the conditions specified. The zeta potential describes the potential at the boundary between bulk solution and the region of hydrodynamic shear or diffuse layer. The term is synonymous with “electrokinetic potential” because it is the potential of the particles which acts outwardly and is responsible for the particle's electrokinetic behavior.

The one or more cationic lipids in the cationic liposomal formulation are selected from the group consisting of N-[1-(2,3-dioleoyloxy)propyl]-N,N,N-trimethyl ammonium salts, such as N-[1-(2,3-dioleoyloxy)propyl]-N,N,N-trimethyl ammonium salt (DOTAP); dimethyldioctadecyl ammonium bromide (DDAB); 1,2-diacyloxy-3-trimethylammonium propanes, including for example, dioleoyl, dimyristoyl, dilauroyl, dipalmitoyl, and distearoyl, and including those with two different acyl chain linked to the glycerol backbone); N-[1-(2,3-dioloyloxy)propyl]-N,N-dimethyl amine (DODAP); 1,2-diacyloxy-3-dimethylammonium propanes, including for example, dioleoyl, dimyristoyl, dilauroyl, dipalmitoyl, and distearoyl, and including those with two different acyl chain linked to the glycerol backbone; N-[1-(2,3-dioleyloxy)propyl]-N,N,N-trimethylammonium chloride (DOTMA); 1,2-dialkyloxy-3-dimethylammonium propanes, including for example dioleyl, dimyristyl, dilauryl, dipalmityl, and distearyl and including those with two different alkyl chain linked to the glycerol backbone; dioctadecylamidoglycylspermine (DOGS); 3β-[N—(N′,N′-dimethylamino-ethane)carbamoyl]cholesterol (DC-Chol); 2,3-dioleoyloxy-N-(2-(sperminecarboxamido)-ethyl)-N,N-dimethyl-1-propanam-inium trifluoro-acetate (DOSPA); β-alanyl cholesterol; cetyl trimethyl ammonium bromide (CTAB); diC14-amidine; N-tert-butyl-N′-tetradecyl-3-tetradecylamino-propionamidine; 14Dea2; N-(alpha-trimethylammonioacetyl)didodecyl-D-glutamate chloride (TMAG); O,O′-ditetradecanoyl-N-(trimethylammonio-acetyl)diethanolamine chloride; 1,3-dioleoyloxy-2-(6-carboxy-spermyl)-propylamide (DOSPER); N,N,N′,N′-tetramethyl-N,N′-bis(2-hydroxylethyl)-2,3-dioleoyloxy-1,4-butan-ediammonium iodide; 1-[2-(acyloxy)ethyl]-alkyl(alkenyl)-3-(2-hydroxyethyl)-imidazolinium chloride derivatives, such as 1-[2-(9(Z)-octadecenoyloxy)ethyl]-2-(8(Z)-heptadecenyl-3-(2-hydroxyethyl)-imidazolinium chloride (DOTIM) and 1-[2-(hexadecanoyloxy)ethyl]-2-pentadecyl-3-(2-hydroxyethyl)imidazolinium chloride (DPTIM); 2,3-dialkyloxypropyl quaternary ammonium compound derivatives, containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimethyl-hydroxypropyl ammonium bromide (DORIE-HP), 1,2-dioleyloxypropyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE); cationic esters of acyl carnitines; and cationic triesters of phospahtidylcholine, for example, 1,2-diacyl-sn-glycerol-3-ethylphosphocholines, in which the hydrocarbon chains are saturated or unsaturated and branched or unbranched with a chain length from C₁₂ to C₂₄, and the two acyl chains may or may not be identical.

Optionally, the liposomal preparation comprises one or more neutral and/or anionic lipids. The neutral and anionic lipids are selected from sterols or lipids such as cholesterol, phospholipids, lysolipids, lysophospholipids, sphingolipids, or pegylated lipids with a neutral or negative net change. In particular embodiments, the neutral and anionic lipids include: phosphatidylserine; phosphatidylglycerol; phosphatidylinositol; fatty acids; sterols containing a carboxylic acid group for example, cholesterol; 1,2-diacyl-sn-glycero-3-phosphoethanolamines, including DOPE; 1,2-diacyl-glycero-3-phosphocholines; and sphingomyelin. The fatty acids linked to the glycerol backbone have various length and number of double bonds. Phospholipids can have two different fatty acids. In embodiments, the neutral and/or anionic lipids are in the liquid crystalline state at room temperature and they are miscible with the used cationic lipid, in the ratio as they are applied. The neutral and/or anionic lipids and the cationic lipids can form a uniform phase and no phase separation or domain formation occurs. In embodiments, the neutral lipid is DOPC (1,2-dioleoyl-sn-glycero-3-phosphocholine).

In embodiments, the cationic liposomal formulation includes taxanes. As used herein, the term “taxane” herein refers to a class of antineoplastic agents having the function of binding microtubules which inhibit cell division and having a structure that includes the taxane ring structure and a stereospecific side chain that is required for cytostatic activity. The term taxane also includes a variety of known derivatives, such as hydrophilic derivatives and hydrophobic derivatives. Taxane derivatives include galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. Examples of taxanes include paclitaxel, docetaxel, and carbazitaxel.

The term “paclitaxel” includes analogues, formulations, and derivatives such as, for example, docetaxel (Taxotere, a formulation of docetaxel), 10-desacetyl analogs of paclitaxel and 3′N-desbenzoyl-3 N-t-butoxycarbonyl analogs of paclitaxel. Paclitaxels can be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis). Paclitaxel refers not only to the common chemically available form of paclitaxel (e.g. Taxol®), but also analogs (e.g., Taxotere, as noted above) and paclitaxel conjugates (e.g., paclitaxel-PEG, paclitaxel-dextran, or paclitaxel-xylose).

The term “derivative” refers to a compound derived from some other compound while maintaining its general structural features. Derivatives may be obtained for example by chemical functionalization or derivatization.

The term “liposomal paclitaxel” or “lipid complexed paclitaxel” refers to a liposomal preparation. A specific liposomal paclitaxel formulation is EndoTAG®-1. The manufacture of such a formulation is disclosed in WO 2004/002468, which is herein incorporated by reference. EndoTAG®-1 is a liposomal preparation with a mole ratio of 50:47:3 mole % of DOTAP, DOPC and paclitaxel.

Due to the steric shape and the amphiphilic nature of the lipids, self-assembly leads to the formation of lipid bilayers (membranes), in which the hydrophobic alkyl chains are oriented toward each other and the polar head groups are oriented toward the aqueous phase. These membranes are organized as spherical vesicles, so-called liposomes. Because of the presence of cationic (positively charged) lipid molecules in the bilayer membrane, the liposomes are cationic. EndoTAG®-1 is delivered as a lyophilized powder for solution for infusion. It is reconstituted with water for injection prior to application. The resulting solution consists of small liposomal vesicles with an intensity weighted average particle size <300 nm.

The cationic liposomal formulation described herein includes one or more cationic lipids, one or more neutral lipids, and paclitaxel. In embodiments, the cationic lipid is DOTAP; the neutral lipid is DOPC. The mole ratio of cationic lipids, neutral lipids, and taxanes is in the range of about 40 to 60 cationic lipids, about 39 to 55 neutral lipids, and about 1 to 5 paclitaxel. In particular embodiments, the cationic liposomal formulation includes DOTAP, DOPC, and paclitaxel in a mole ratio of about 50:47:3.

The cationic liposomal formulation can include one or more carriers. As used herein, the term “carrier” refers to a diluent, adjuvant, excipient, or vehicle which is suitable for administering a diagnostic or therapeutic agent. The term also refers to a pharmaceutically acceptable carrier that contain, complexes or is otherwise associated with an agent to facilitate the transport of such an agent to its intended target site. Carriers include those known in the art, such as liposomes, polymers, lipid complexes, serum albumin, antibodies, cyclodextrins, dextrans, chelates, or other supramolecular assemblies.

The formulations, in particular the cationic liposomal formulation, disclosed herein can be provided in a dry, dehydrated, or lyophilized form. Prior to administration, the formulation can be hydrated in pharmaceutical grade water or saline or another suitable liquid, preferably comprising physiologically acceptable carriers such as a buffer.

The formulations disclosed herein can be provided in the form of kits. In embodiments, the kit can include a cationic liposomal formulation and one or more active agents described herein. The one or more active agents can be a chemotherapeutic agent. In particular embodiments, the non-liposomal formulation in the kit includes a taxane, such as paclitaxel, and the active agent is gemcitabine. The kits provided herein can also include a container and/or reagents for preparing the formulations for administration. As an example, the cationic liposomal formulation can be in a dehydrated form that can be reconstituted by hydration.

As used herein, the term “combination” or “co-administration” refers to an administration schedule that is synchronous, serial, overlapping, alternating, parallel, or any other treatment schedule in which the various active agents or therapies are administered as part of a single treatment regimen, prescription or indication or in which the time periods during which the various agents or therapies that are administered otherwise partially or completely coincide.

Depending on the duration of the treatment and on the observed side effects, the administration of the formulations can also be omitted for at least one week or several weeks during the treatment period.

In embodiments, the methods described herein include administering the cationic liposomal formulation in a single dose of from about 1 mg/m² to about 60 mg/m².

As used herein, the unit mg/m², refers to mg of active agent, for example paclitaxel, per m² body surface area (bsa) of the subject.

As used herein, the unit mg/kg body weight of a subject or mg/kg refers to mg of active agent, for example paclitaxel, per kg body weight (bw) of a subject.

In embodiments, on an average, a human subject has a body surface area of about 1.84 m². Thus, for an average person of 70 kg body weight and 172 cm height, values for single doses, monthly doses, etc. which are in mg/kg body weight (bw) may be converted for human applications to corresponding values of in mg/m² human body surface area (bs) by multiplication with a species-specific factor according to known methods. Similarly, doses in mg/m² bs of a human subject can be converted to mg/kg bw of a human subject.

Provided herein are methods for treating a subject having pancreatic cancer and the subject is refractory or resistant to a first-line or second-line therapy. In embodiments, the first-line or second-line therapy includes administering an antineoplastic agent, a combination therapy, gemcitabine-based therapy, a fluorouracil-based combination therapy, a growth factor inhibitor therapy, or an antimitotic agent therapy.

In embodiments, the first-line or second-line therapy includes administering one or more antineoplastic agents comprising fluorouracil, bleomycin, bortezomib, carboplatin, cisplatin, cytarabine, docetaxel, doxorubicin, elmustin, erlotinib, etoposide, gemcitabine, idarubicin, imatinib, lomustine, methotrexate, mitomycin, mitoxantrone, oxaliplatin, paclitaxel, pemetrexed, sunitinib, topotecan, treosulfan, vemurafenib, vinblastine, vincristine, vindesine, or vinorelbine.

In some embodiments, the subject is refractory or resistant to a gemcitabine-based therapy, such as gemcitabine-based combination therapy or gemcitabine monotherapy.

In some embodiments, the subject is refractory or resistant to a combination of albumin-bound paclitaxel and gemcitabine a combination of erlotinib and gemcitabine, a combination of capecitabine and gemcitabine, or a combination of cisplatin and gemcitabine.

In some embodiments, the subject is refractory or resistant to a fluorouracil-based combination therapy.

In some embodiments, the subject is refractory or resistant to a combination of oxaliplatin, leucovorin, irinotecan and fluorouracil or a combination of leucovorin, liposomal irinotecan and fluorouracil.

In some embodiments, the subject is refractory or resistant to a growth factor inhibitor such as erlotinib, cetuximab, gefinitib, imatinib, panitumumab, sunitinib, or vemurafenib.

In some embodiments, the subject is refractory or resistant to an antimitotic agent such as paclitaxel, docetaxel, vinblastine, vincristine, vindesine, or vinorelbine.

In some embodiments, the subject is in need of a second-line treatment and has previously been treated with an intravenous infusion of about 70 mg/m² to 100 mg/m² oxaliplatin followed by an intravenous infusion of about 300 mg/m² to 500 mg/m² leucovorin concomitantly with an intravenous infusion of about 90 mg/m² to 270 mg/m² irinotecan, followed by an intravenous bolus of about 300 mg/m² to 800 mg/m² fluorouracil and an intravenous infusion of about 1200 mg/m² to 3600 mg/m² fluorouracil.

In some embodiments, the subject has previously been treated with an intravenous infusion of about 85 mg/m² oxaliplatin followed by an intravenous infusion of about 400 mg/m² leucovorin concomitantly with an intravenous infusion of about 180 mg/m² irinotecan, followed by an intravenous bolus of about 400 mg/m² fluorouracil and an intravenous infusion of about 2400 mg/m² fluorouracil.

In some embodiments, the subject is in need of a second-line or third-line treatment and has previously been treated with one or more antineoplastic agents without administering a pulsed dose.

In some embodiments, the subject is in need of a second-line or third-line treatment and has previously been treated with an intravenous infusion of about 70 mg/m² to 100 mg/m² oxaliplatin followed by an intravenous infusion of about 300 mg/m² to 500 mg/m² leucovorin concomitantly with an intravenous infusion of about 90 mg/m² to 180 mg/m² irinotecan, followed by an intravenous infusion of about 1200 mg/m² to 3600 mg/m² fluorouracil.

In some embodiments, the subject is in need of a second-line or third-line treatment and has previously been treated with an intravenous infusion of about 85 mg/m² oxaliplatin followed by an intravenous infusion of about 400 mg/m² leucovorin concomitantly with an intravenous infusion of about 130 mg/m² to 150 mg/m² irinotecan, followed by an intravenous infusion of about 2400 mg/m² fluorouracil.

The methods described herein include administering a cationic liposomal formulation including taxane, such as paclitaxel or a derivative thereof, and a further antineoplastic agent such as gemcitabine.

In the methods described herein, the cationic liposomal formulation is administered in a dose of from about 1 mg/m² to about 50 mg/m², about 25 mg/m² to about 50 mg/m², about 10 mg/m² to about 25 mg/m², or from about 11 mg/m² to about 22 mg/m² of the body surface area (bsa) of the subject. In particular embodiments, the cationic liposomal formulation is administered at a dose of about 1 mg/m², about 2.5 mg/m², about 5 mg/m², about 7.5 mg/m², 11 mg/m², about 22 mg/m², about 25 mg/m², about 28 mg/m², about 31 mg/m², about 33 mg/m², about 35 mg/m², about 38 mg/m², about 41 mg/m², about 44 mg/m², or about 47 mg/m² of the bsa of the subject.

In embodiments, gemcitabine is administered at a dose from about 100 mg/m² to about 1500 mg/m², about 100 mg/m² to about 500 mg/m², about 500 mg/m² to about 1500 mg/m², about 600 mg/m² to about 1400 mg/m², about 700 mg/m² to about 1300 mg/m², about 800 mg/m², or about 1250 mg/m² bsa of the subject. In particular, gemcitabine is administered at a dose of about 500 mg/m² or 1000 mg/m².

In embodiments, the cationic liposomal formulation is administered twice weekly, and gemcitabine is administered once weekly.

In embodiments, the cationic liposomal formulation comprising paclitaxel is administered on days 1, 4, 8, 11, 15, 18, 22, 25, 29, 32, 36, 39, 43 and 46 at a dose of about 1 to 60 mg/m² and gemcitabine at a dose of about 300 to 1500 mg/m² is administered on days 4, 11, 18, 25, 32, 39 and 46 of a treatment cycle of seven weeks.

In embodiments, methods for treating pancreatic cancer are provided, wherein the methods comprise a first treatment cycle, which is followed by one or more subsequent treatment cycles. The first treatment cycle is a period of seven weeks, while each subsequent treatment cycle is a period of three weeks. In the first treatment cycle, the cationic liposomal formulation comprising paclitaxel at a dose of about 1 to 60 mg/m² is administered on days 1, 4, 8, 11, 15, 18, 22, 25, 29, 32, 36, 39, 43 and 46 and gemcitabine at a dose of about 300 to 1500 mg/m² is administered on days 4, 11, 18, 25, 32, 39 and 46. A dosing interval between the first treatment cycle and the subsequent treatment cycle is one week. In the subsequent treatment cycles, the cationic liposomal formulation comprising paclitaxel at a dose of about 1 to 60 mg/m² is administered on days 1, 4, 8, 11, 15 and 18 and gemcitabine at a dose of about 300 to 1500 mg/m² is administered on days 4, 11 and 18. A dosing interval between two subsequent treatment cycles is one week.

In some embodiments, the methods comprise administering about 1 mg/m² to about 60 mg/m² paclitaxel in the cationic liposomal formulation and about 300 mg/m² to about 1500 mg/m² gemcitabine to the subject.

In some embodiments, the methods comprise administering about 11 mg/m² to about 22 mg/m² paclitaxel in the cationic liposomal formulation and about 500 mg/m² to about 1000 mg/m² gemcitabine to the subject.

In some embodiments, the cationic liposomal formulation is administered to the subject at a rate of 0.5 mL/min for first 15 minutes, followed by a rate of 1.0 mL/min for second 15 minutes, and followed by a rate of 1.5 mL/min after 30 minutes.

In embodiments, gemcitabine can be applied at a lower weekly dose compared to that in the standard of care for pancreatic cancer (1000 mg/m²). In some embodiments, gemcitabine is administered at a dose of about 500 mg/m², 550 mg/m², 600 mg/m², 650 mg/m², 700 mg/m², 750 mg/m², or 800 mg/m².

The continued administration of lower doses once or twice weekly is at least as effective as the administration of a single high dose or frequent low dose administration interrupted by pause intervals. Depending on the effectiveness of the dosage regimen, the doses of the formulations and the dosing intervals may remain constant, increased, or decreased during the treatment period.

In embodiments, the method disclosed herein is used after a neoadjuvant therapy which refers to a treatment given as a first step to shrink a tumor before the main treatment, for example surgery, is given. A neoadjuvant therapy includes but is not limited to chemotherapy, radiation therapy, and hormone therapy. As an example, the treatment of pancreatic cancer includes a neoadjuvant therapy including administering FOLFIRINOX, followed by surgery, which is followed by a method described herein.

The methods disclosed herein are characterized by selective targeting, improved efficacy, reduced adverse side effects as compared to conventional chemotherapy, reduced disease related pain, improved quality of life, stabilization of body weight during treatment, and synergistic effects with other therapy regimens.

In embodiments, the methods include inhibiting the growth of pancreatic cancer cells that are refractory or resistant to one or more (a combination of) antineoplastic agents, for example multidrug resistant (MDR) cells. The antineoplastic agents are fluorouracil, bleomycin, bortezomib, carboplatin, cisplatin, cytarabine, docetaxel, doxorubicin, elmustin, erlotinib, etoposide, gemcitabine, idarubicin, imatinib, lomustine, methotrexate, mitomycin, mitoxantrone, oxaliplatin, paclitaxel, pemetrexed, sunitinib, topotecan, treosulfan, vemurafenib, vinblastine, vincristine, vindesine and vinorelbine. Examples of MDR pancreatic cancer cells include PAXF 546, PAXF 1986, PACF 1998, PACF 2005, PAXF 2035, PAXF 2059, PAXF CAPAN-2, and PACF PANC-1.

In embodiments, the MDR pancreatic cancer cells are in vitro cells, in vivo cells, ex vivo cells, or cells obtained from a xenograft.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. In particular embodiments, lack of a material effect is evidenced by lack of a statistically-significant reduction in the embodiment's ability to kill pancreatic cancer cells in vitro or in vivo.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of 20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The subject matter described above is provided by way of illustration only and should not be construed as limiting. Various modifications and changes may be made to the subject matter described herein without following the example embodiments and applications illustrated and described, and without departing from the true spirit and scope of the present invention, which is set forth in the following claims.

The following examples illustrate exemplary methods provided herein. These examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### EXEMPLARY EMBODIMENTS

The following are exemplary embodiments:

1. A method of treating refractory or resistant pancreatic cancer, wherein the method comprises administering to a subject in need thereof (a) a cationic liposomal formulation comprising one or more cationic lipids and a therapeutically effective amount of paclitaxel, and (b) a therapeutically effective amount of gemcitabine.

2. The method of embodiment 1, wherein the refractory or resistant pancreatic cancer is refractory or resistant to one or more antineoplastic agents comprising fluorouracil, bleomycin, bortezomib, carboplatin, cisplatin, cytarabine, docetaxel, doxorubicin, elmustin, erlotinib, etoposide, gemcitabine, idarubicin, imatinib, lomustine, methotrexate, mitomycin, mitoxantrone, oxaliplatin, paclitaxel, pemetrexed, sunitinib, topotecan, treosulfan, vemurafenib, vinblastine, vincristine, vindesine, or vinorelbine.

3. The method of embodiment 1 or 2, wherein the refractory or resistant pancreatic cancer is refractory or resistant to a fluorouracil-based combination therapy.

4. The method of any one of embodiments 1-3, wherein the refractory or resistant pancreatic cancer is refractory or resistant to a combination of oxaliplatin, leucovorin, and/or irinotecan, and fluorouracil.

5. The method of any one of embodiments 1-4, wherein the refractory or resistant pancreatic cancer is refractory or resistant to a gemcitabine-based combination therapy.

6. The method of any one of embodiments 1-4, wherein the refractory or resistant pancreatic cancer is refractory or resistant to a growth factor inhibitor.

7. The method of any one of embodiments 1-6, wherein the growth factor inhibitor is erlotinib, cetuximab, gefinitib, imatinib, panitumumab, sunitinib, or vemurafenib.

8. The method of any one of embodiments 1-7, wherein the refractory or resistant pancreatic cancer is refractory or resistant to an antimitotic agent.

9. The method of any one of embodiments 1-8, wherein the antimitotic agent is paclitaxel, docetaxel, vinblastine, vincristine, vindesine, or vinorelbine.

10. The method of any one of embodiments 1-9, wherein the subject has previously been treated with an intravenous infusion of about 70 mg/m² to 100 mg/m² oxaliplatin followed by an intravenous infusion of about 300 mg/m² to 500 mg/m² leucovorin concomitantly with an intravenous infusion of about 90 mg/m² to 270 mg/m² irinotecan, followed by an intravenous bolus of about 300 mg/m² to 800 mg/m² fluorouracil and an intravenous infusion of about 1200 mg/m² to 3600 mg/m² fluorouracil.

11. The method of any one of embodiments 1-10, wherein the subject has previously been treated with an intravenous infusion of about 85 mg/m² oxaliplatin followed by an intravenous infusion of about 400 mg/m² leucovorin concomitantly with an intravenous infusion of about 180 mg/m² irinotecan, followed by an intravenous bolus of about 400 mg/m² fluorouracil and an intravenous infusion of about 2400 mg/m² fluorouracil.

12. The method of any one of embodiments 1-11, wherein the subject has previously been treated with one or more antineoplastic agent without administering a pulsed dose.

13. The method of any one of embodiments 1-12, wherein the subject has previously been treated with an intravenous infusion of about 70 mg/m² to 100 mg/m² oxaliplatin followed by an intravenous infusion of about 300 mg/m² to 500 mg/m² leucovorin concomitantly with an intravenous infusion of about 90 mg/m² to 180 mg/m² irinotecan, followed by an intravenous infusion of about 1200 mg/m² to 3600 mg/m² fluorouracil.

14. The method of any one of embodiments 1-13, wherein the subject has previously been treated with an intravenous infusion of about 85 mg/m² oxaliplatin followed by an intravenous infusion of about 400 mg/m² leucovorin concomitantly with an intravenous infusion of about 130 mg/m² to 150 mg/m² irinotecan, followed by an intravenous infusion of about 2400 mg/m² fluorouracil.

15. The method of any one of embodiments 1-14, wherein the method comprises administering about 1 mg/m² to about 60 mg/m² paclitaxel in the cationic liposomal formulation and about 300 mg/m² to about 1500 mg/m² gemcitabine to the subject.

16. The method of any one of embodiments 1-15, wherein the method comprises administering about 11 mg/m² to about 22 mg/m² paclitaxel in the cationic liposomal formulation and about 500 mg/m² to about 1000 mg/m² gemcitabine to the subject.

17. The method of any one of embodiments 1-16, wherein the cationic liposomal formulation is administered twice weekly, and gemcitabine is administered once weekly.

18. The method of any one of embodiments 1-17, wherein the cationic liposomal formulation is administered on days 1, 4, 8, 11, 15, 18, 22, 25, 29, 32, 36, 39, 43, and 46 and gemcitabine is administered on days 4, 11, 18, 25, 32, 39, and 46 of a first treatment cycle of seven weeks.

19. The method of any one of embodiments 1-18, wherein the first treatment cycle is followed by one or more subsequent treatment cycles, the cationic liposomal formulation is administered on days 1, 4, 8, 11, 15, and 18 and gemcitabine is administered on days 4, 11, and 18 of a subsequent treatment cycle of three weeks, and a dosing interval between a first treatment cycle and a subsequent treatment cycle or between two subsequent treatment cycles is one week.

20. The method of any one of embodiments 1-19, wherein the cationic liposomal formulation is administered at a rate of 0.5 mL/min for first 15 minutes, followed by a rate of 1.0 mL/min for second 15 minutes, and followed by a rate of 1.5 mL/min after 30 minutes.

21. A method of treating refractory or resistant pancreatic cancer, wherein the method comprises administering to a subject in need thereof a cationic liposomal formulation comprising one or more cationic lipids and a therapeutically effective amount of paclitaxel.

22. The method of embodiment 21, wherein the refractory or resistant pancreatic cancer is refractory or resistant to one or more antineoplastic agents comprising fluorouracil, bleomycin, bortezomib, carboplatin, cisplatin, cytarabine, docetaxel, doxorubicin, elmustin, erlotinib, etoposide, gemcitabine, idarubicin, imatinib, lomustine, methotrexate, mitomycin, mitoxantrone, oxaliplatin, paclitaxel, pemetrexed, sunitinib, topotecan, treosulfan, vemurafenib, vinblastine, vincristine, vindesine, or vinorelbine.

23. The method of embodiment 21 or 22, wherein the refractory or resistant pancreatic cancer is refractory or resistant to a fluorouracil-based combination therapy.

24. The method of any one of embodiments 21-23, wherein the refractory or resistant pancreatic cancer is refractory or resistant to a combination of oxaliplatin, leucovorin, and/or irinotecan, and fluorouracil.

25. The method of any one of embodiments 21-24, wherein the refractory or resistant pancreatic cancer is refractory or resistant to a gemcitabine-based combination therapy.

26. The method of any one of embodiments 21-25, wherein the refractory or resistant pancreatic cancer is refractory or resistant to a growth factor inhibitor.

27. The method of any one of embodiments 21-26, wherein the growth factor inhibitor is selected from a group consisting of erlotinib, cetuximab, gefinitib, imatinib, panitumumab, sunitinib, or vemurafenib.

28. The method of any one of embodiments 21-27, wherein the refractory or resistant pancreatic cancer is refractory or resistant to an antimitotic agent.

29. The method of any one of embodiments 21-28, wherein the antimitotic agent is selected from a group consisting of paclitaxel, docetaxel, vinblastine, vincristine, vindesine, or vinorelbine.

30. The method of any one of embodiments 1-29, wherein the cationic liposomal formulation comprises a cationic lipid from about 30 mole % to about 99.9 mole %, paclitaxel in an amount of at least 0.1 mole % and a neutral or an anionic lipid in an amount of 30 mole % to 55 mole %, and the cationic liposomal formulation has a positive zeta potential in about 0.05 M KCl solution at about pH 7.5 at room temperature.

31. The method of any one of embodiments 1-30, wherein the cationic liposomal formulation comprises DOTAP, DOPC, and paclitaxel.

32. The method of any one of embodiments 1-31, wherein the cationic liposomal formulation comprises DOTAP, DOPC, and paclitaxel in a mole ratio of about 50:47:3.

33. The method of any one of embodiments 1-32, wherein the cationic lipid is N-[1-(2,3-dioleoyloxy)propyl]-N,N,N-trimethyl ammonium salt (DOTAP); dimethyldioctadecyl ammonium bromide (DDAB); 1,2-diacyloxy-3-trimethylammonium propane N-[1-(2,3-dioloyloxy)propyl]-N, N-dimethyl amine (DODAP); 1,2-diacyloxy-3-dimethylammonium propane; N-[1-(2,3-dioleyloxy)propyl]-N,N,N-trimethylammonium chloride (DOTMA); 1,2-dialkyloxy-3-dimethylammonium propane; dioctadecylamidoglycylspermine (DOGS); 3β-[N—(N′,N′-dimethylamino-ethane)carbamoyl]cholesterol (DC-Chol); 2,3-dioleoyloxy-N-(2-(sperminecarboxamido)-ethyl)-N, N-dimethyl-1-propanaminium trifluoroacetate (DOSPA); β-alanyl cholesterol; cetyl trimethyl ammonium bromide (CTAB); diC14-amidine; N-tert-butyl-N′-tetradecyl-3-tetradecylamino-propionamidine; 14Dea2; N-(alpha-trimethylammonioacetyl)didodecyl-D-glutamate chloride (TMAG); O,O′-ditetradecanoyl-N-(trimethylammonioacetyl)diethanolamine chloride; 1,3-dioleoyloxy-2-(6-carboxy-spermyl)-propylamide (DOSPER); N,N,N′,N′-tetrarnethyl-N,N′-bis(2-hydroxylethyl)-2,3-dioleoyloxy-1,4-butanediarnmonium iodide; 1-[2-(acyloxy)ethyl]2-alkyl (alkenyl)-3-(2-hydroxyethyl)-imidazolinium chloride; 1,2-dioleoyl-3-dimethyl-hydroxyethylammonium bromide (DORI); 1,2-dioleyloxypropyl-3-dimethylhydroxyethylammonium bromide (DORIE); 1,2-dioleyloxypropyl-3-dimethylhydroxypropylammonium bromide (DORIE-HP); 1,2-dioleyloxypropyl-3-dimethylhydroxybutylammonium bromide (DORIE-HS); 1,2-dioleyloxypropyl-3-dimethylhydroxypentylammonium bromide (DORIE-Hpe); 1,2-dimyristyloxypropyl-3-dimethylhydroxylethylammonium bromide (DMRIE); 1,2-dipalmityloxypropyl-3-dimethylhydroxyethylammonium bromide (DPRIE); 1,2-disteryloxypropyl-3-dimethylhydroxyethylammonium bromide (DSRIE); or 1,2-diacyl-sn-glycerol-3-ethylphosphocholine.

34. The method of any one of embodiments 1-33, wherein the 1-[2-(acyloxy)ethyl]2-alkyl (alkenyl)-3-(2-hydroxyethyl)-imidazolinium chloride is 1-[2-(9(Z)-octadecenoyloxy)ethyl]-2-(8(Z)-heptadecenyl-3-(2-hydroxyethyl)-imidazoliniumchloride (DOTIM) or 1-[2-(hexadecanoyloxy)ethyl]-2-pentadecyl-3-(2-hydroxyethyl)imidazolinium chloride (DPTIM).

35. The method of any one of embodiments 1-34, wherein the neutral lipid is cholesterol, phospholipid, lysolipid, sphingolipid, or pegylated lipid with a neutral charge.

36. The method of any one of embodiments 1-35, wherein the neutral lipid is lysophospholipid.

37. The method of any one of embodiments 1-36, wherein the neutral lipid is 1,2-diacyl-sn-glycero-3-phosphoethanolamine, 1,2-diacyl-sn-glycero-3-phosphocholine, or sphingomyelin.

38. The method of any one of embodiments 1-37, wherein 1,2-diacyl-sn-glycero-3-phosphoethanolamine is 1,2-dioleoyl-sn-glycero-3-phosphoethanolamine (DOPE).

39. The method of any one of embodiments 1-38, wherein 1,2-diacyl-sn-glycero-3-phosphocholine is 1,2-dioleoyl-sn-glycero-3-phosphocholine (DOPC).

40, The method of any one of embodiments 1-39, wherein the cationic liposomal formulation and the gemcitabine are administered simultaneously but separately.

41, The method of any one of embodiments 1-40, wherein the cationic liposomal formulation and the gemcitabine are administered sequentially.

42. A method of any one of embodiments 1-41, wherein the method includes inhibiting the growth of multidrug resistant (MDR) pancreatic cells comprising administering to MDR pancreatic cells a cationic liposomal formulation comprising one or more cationic lipids and a therapeutically effective amount of paclitaxel.

43. The method of any one of embodiments 1-42, wherein the method further comprises administering a therapeutically effective amount of gemcitabine.

44. The method of any one of embodiments 1-43, wherein the cationic liposomal formulation and the therapeutically effective amount of gemcitabine are administered simultaneously, but separately, or are administered sequentially.

45. The method of any one of embodiments 1-44, wherein the method is preceded by a neoadjuvant therapy.

46. A method of treating pancreatic cancer, wherein the method comprises administering a neoadjuvant therapy followed by administering:
(i) a cationic liposomal formulation comprising one or more cationic lipids and a therapeutically effective amount of paclitaxel; or
(ii)) two formulations: (a) a cationic liposomal formulation comprising one or more cationic lipids and a therapeutically effective amount of paclitaxel, and (b) a therapeutically effective amount of gemcitabine.

47. The method of embodiment 45 or 46, wherein the method further includes performing surgery after the neoadjuvant therapy and before administering the cationic liposomal formulation or the two formulations.

### EXAMPLE

### Example 1: Multidrug Resistant Cells

Pancreatic cancer cell lines resistant to one or more agents such as fluorouracil, bleomycin, bortezomib, carboplatin, cisplatin, cytarabine, docetaxel, doxorubicin, elmustin, erlotinib, etoposide, gemcitabine, idarubicin, imatinib, lomustine, methotrexate, mitomycin, mitoxantrone, oxaliplatin, paclitaxel, pemetrexed, sunitinib, topotecan, treosulfan, vemurafenib, vinblastine, vincristine, vindesine and/or vinorelbine were selected for this study. The cell lines selected include PAXF 546, PAXF 1986, PACF 1998, PACF 2005, PAXF 2035, PAXF 2059, PAXF CAPAN-2, PAXF HPAC, and PACF PANC-1. These cells lines were treated with EndoTAG®-1 (cationic liposomal paclitaxel formulation) and empty cationic liposomes (used for control), which were manufactured according to the method of EndoTAG®-1.

One vial of EndoTAG-1® containing 6.4 mg paclitaxel was dissolved in 23 mL water for injection by gentle shaking for 20 times until no undissolved powder is observed. The vial was stored at room temperature for at least 30 minutes to allow for complete reconstitution. The vial was repeatedly shaken after the storage period. Temperature did not exceed 30° C. at any time.

Stock solutions of EndoTAG®-1 and empty liposomes, each containing 300 μM in 10.5% trehalose were prepared right before adding to the assay wells. In a first step 1:2 dilution was prepared to reach nominal paclitaxel concentration of 128 mg/L. EndoTAG-1® and empty liposomes were next serially diluted in half-log steps with 10.5% trehalose on an intermediate dilution plate, followed by a further 1:10 dilution with 10.5% trehalose. Finally, 10 μL taken from this dilution plate was transferred to 140 L/well of the cell culture plate. EndoTAG-1® and empty liposomes were tested at 10 concentrations in triplicate in half-log steps up to 1 μM. Thus, final concentration of trehalose in each assay well was 0.7% w/v.

Cell lines were routinely passaged once or twice weekly and maintained in culture for up to 20 passages. Cells were grown at 37° C. in a humidified atmosphere with 5% CO₂ in RPMI 1640 medium (25 mM HEPES, with L-glutamine, #FG1385, Biochrom, Berlin, Germany) supplemented with 10% (v/v) fetal calf serum (Sigma, Taufkirchen, Germany) and 0.1 mg/mL gentamicin (Life Technologies, Karlsruhe, Germany).

The CellTiter-Blue® Cell Viability Assay (#G8081, Promega) was used to investigate anti-tumor activity. Cells were harvested from exponential phase cultures, counted and plated in 96-well flat-bottom microtiter plates at a cell density of 8,000-12,000 cells/well depending on the cell line's growth rate. After a 24 h recovery period to allow the cells to resume exponential growth, 10 μL of culture medium (four control wells/plate) or of culture medium with EndoTAG®-1 or culture medium with empty liposomes was added to the cells. EndoTAG®-1 was applied at 10 concentrations in triplicate in half-log increments up to 1 μM and treatment continued for three days. After treatment of cells, 20 μL/well CellTiter-Blue® reagent was added. Following an incubation period of up to four hours, fluorescence (FU) was measured by using the Enspire Multimode Plate Reader (excitation λ=531 nm, emission λ=615 nm). For calculations, the mean values of triplicate data were used. Pancreatic cancer cell lines resistant to one or more antineoplastic agents (see Table 2) were treated with EndoTAG®-1 and empty liposomes. IC₅₀ of EndoTAG®-1 are shown in Table 1.

Table 1 shows that EndoTAG®-1 is effective in inhibiting the growth of eight MDR pancreatic cells. No growth inhibitory effect was observed with MDR pancreatic cell lines treated with empty liposomes.

TABLE 1
Relative IC₅₀ (μM)

treatment

| Cell line | EndoTAG-1 | Empty liposomes |
|---|---|---|
| PAXF 546 | 0.005 | 1.0 |
| PAXF 1986 | 0.011 | 1.0 |
| PAXF 1998 | 0.039 | 1.0 |
| PAXF 2005 | 0.006 | 1.0 |
| PAXF 2035 | 0.032 | 1.0 |
| PAXF 2059 | 0.004 | 1.0 |
| PAXF CAPAN-2 | 0.013 | 1.0 |
| PAXF PANC-1 | 0.008 | 1.0 |

TABLE 2
Cell lines echibiting drug resistance (IC₅₀) (μM)

| | Cell line | | | | | | | | |
|---|---|---|---|---|---|---|---|---|---|
| | PAXF | PAXF | PAXF | PAXF | PAXF | PAXF | PAXF | PAXF | PAXF |
| Drug | CAPAN-2 | HPAC | PANC-1 | 546 | 1986 | 1998 | 2005 | 2035 | 2059 |
| 5-Fluorouracil | | | 3.228 | 1.246 | 2.253 | 7.475 | 10.328 | 105.27 | |
| Bleomycin sulfate | | | 0.149 | 10.705 | | | | | |
| Bortezomib | | | 0.018 | 0.005 | | | | | |
| Carboplatin | | | 15.634 | 17.082 | | | | | |
| Cisplatin | 62.143 | 31.722 | 7.441 | 11.513 | 4.831 | 75.726 | 10.768 | 46.021 | 40.966 |
| Cytarabine | | | 0.158 | 4.637 | | | | | |
| Docetaxel | | | 0.000 | 0.104 | 0.006 | 0.015 | 0.100 | 0.004 | 0.100 |
| Doxorubicin HCl | | | 0.049 | 0.134 | 0.059 | 1.232 | 0.251 | 0.795 | 0.131 |
| Elmustin | | | 374.97 | 722.40 | | | | | |
| Erlotinib HCl | | | 113.26 | 46.123 | 100.00 | 99.090 | 52.700 | 83.616 | |
| Etoposide | | | 1.496 | 7.405 | | | | | |
| Gemcitabine HCl | 3.670 | 0.421 | 5.216 | 0.097 | 0.008 | 0.158 | 0.390 | 1.492 | 0.015 |
| Idarubicin | | | 0.012 | 0.131 | | | | | |
| Imatinib, mesylate | | | 36.767 | 20.052 | | | | | |
| Lomustine | | | 43.617 | 142.17 | | | | | |
| Methotrexate | | | 10.000 | 0.066 | | | | | |
| Methotrexate Hydrat | | | 0.038 | 0.129 | | 0.160 | 10.000 | | 0.031 |
| Mitomycin C | | | 1.121 | 0.465 | 0.188 | 0.987 | 0.276 | 0.678 | |
| Mitoxantron 2HCl | | | 0.010 | 0.187 | 0.035 | 0.240 | 0.016 | 0.067 | 0.035 |
| Oxaliplatin | | | 0.994 | 1.240 | 1.606 | 34.622 | 12.475 | 99.179 | |
| Paclitaxel | 0.444 | 0.383 | 0.046 | 0.441 | 0.204 | 0.117 | 0.312 | 0.144 | 0.261 |
| Pemetrexed, dinatrium | | | 12.985 | 0.114 | | | | | |
| Sunitinib malate | | | 8.551 | 7.381 | 4.236 | 13.216 | 7.093 | 2.404 | 16.472 |
| Topotecan HCl | | | 0.042 | 0.080 | | | | | |
| Treosulfan | | | 2.015 | 48.058 | | | | | |
| Vemurafenib | | | 16.078 | 5.329 | 9.337 | 10.768 | 6.753 | 11.432 | 11.392 |
| Vinblastine sulfate | | | 0.001 | 0.071 | | | | | |
| Vincristine sulfate | | | 0.003 | 0.247 | 0.300 | 0.008 | 0.045 | 0.462 | |
| Vindesin sulfate | | | 0.005 | 0.109 | | | | | |
| Vinorelbine bistartrate | | | 0.002 | 0.072 | | | | | |

### Example 2

EndoTAG®-1 plus gemcitabine vs. gemcitabine monotherapy in patients with locally advanced and/or metastatic adenocarcinoma of the pancreas

1.1 Objectives

The objective of the study is to assess the safety, efficacy and quality of life of a combination therapy of EndoTAG®-1 plus gemcitabine vs. gemcitabine monotherapy in patients with locally advanced and/or metastatic adenocarcinoma of the pancreas eligible for second-line therapy after failing first-line therapy with FOLFIRINOX.

1.2 Endpoints

Primary Efficacy Endpoint:

Overall survival time is defined as time from randomization to death from any cause or last day known to be alive.

Secondary Efficacy Endpoints:
1. Progression Free Survival (PFS)
   Progression Free Survival time is defined as the time from randomization to either first observation of progressive disease or occurrence of death.
2. Percentage of subjects with Objective Response (OR) according to Response Evaluation Criteria in Solid Tumors Version 1.1 (RECIST v.1.1) Percentage of subjects with objective response is based on assessment of complete response (CR) or partial response (PR) according to RECIST v.1.1.
3. Duration of Response (DR)
   Duration of Response is defined as the time from the first documentation of objective tumor response (date of the first CR or PR) to objective tumor progression or death due to any cause.
4. Percentage of subjects with disease control according to RECIST v.1.1
   Percentage of subjects with disease control is based on assessment of complete response (CR) or partial response (PR) or stable disease (SD) according to RECIST v.1.1
5. Change from baseline in Quality of Life (QoL) scale
6. Changes from baseline in ECOG performance status
   Eastern Cooperative Oncology Group (ECOG) performance status is used to quantify the functional status of subjects. Number of patients with improvement, steady state, and deterioration at the end of cycle 1 (or at the end of full treatment course) will be evaluated.
7. Pain intensity using a Visual Analog Scale (VAS)
   The VAS is used to assess pain intensity. The score can vary between “0” and “10” wherein, 0=no pain and 10=the worst possible pain.
8. Serum Carcinoma Antigen 19-9 (CA 19-9) response rate
   Responders are defined as subjects with a reduction in CA 19-9 levels by least 50% from baseline to the end of cycle 1 (or end of full treatment course).

Safety Endpoints
1. Incidence and percentage of subjects with treatment-emergent adverse events (TEAEs) during cycle 1 and the full treatment course
2. AEs leading to discontinuation of study medication, interruption of infusion of gemcitabine or EndoTAG®-1, or postponement of subsequent dose of study medication
3. Incidence and percentage of clinically significant abnormal laboratory values during cycle 1 and the full treatment course
4. Incidence and percentage of clinically significant abnormal physical examination and vital signs during cycle 1 and the full treatment course

1.3 Study Design

This is a randomized controlled, open label phase-3 study to evaluate the safety and efficacy of a combination regimen of twice weekly infusions of EndoTAG®-1 (Lipid Complexed Paclitaxel) with weekly administration of gemcitabine compared with gemcitabine monotherapy in subjects with measurable locally advanced and/or metastatic adenocarcinoma of the pancreas who are eligible for second-line therapy after failing first-line therapy with FOLFIRINOX.

Eligible subjects will be randomized to one of the two treatment arms:
Arm A: Treatment with EndoTAG®-1 22 mg/m² twice weekly plus gemcitabine 1000 mg/m² once weekly, for 1 cycle consisting of 7 weeks and 1 week rest followed by subsequent cycles consisting of 3 weeks of treatment and 1 week rest until any one of the following occurs: progressive disease or unacceptable toxicity or withdrawal of patient consent.
Arm B: Treatment with gemcitabine 1000 mg/m² once weekly, for 1 cycle consisting of 7 weeks and 1 week rest followed by subsequent cycles consisting of 3 weeks of treatment and 1 week rest until any one of the following occurs: progressive disease or unacceptable toxicity or withdrawal of patient consent.

The randomization is stratified by
Subjects with locally advanced vs metastatic pancreatic cancer
Subjects with ECOG performance status 0 vs 1

The first treatment cycle is last at least 8 weeks and include 7 weekly (Arm B) or 14 twice weekly (Arm A) treatment visits followed by an EoT visit. Subjects may continue to receive additional cycles of therapy until progressive disease or intolerable toxicity as per clinical judgment of the Investigator.

Tumor response according to RECIST (version 1.1; Eisenhauer et al. 2009) is evaluated on a scheduled basis every 8 weeks (±3 days) from randomization (regardless of the timing of treatment cycles) until disease progression is documented or until the cut-off date of the study, whichever comes earlier. Subjects are monitored regularly for safety parameters, pain and quality of life.

After completing treatment, subjects who diagnosed with progressive disease (PD) are attend up to 6 Follow-up Visits every 8 weeks for 48 weeks, following which subjects will be followed-up by telephone every 8 weeks for survival. Subjects who experienced PD during Treatment Phase are undergo only one safety follow-up visit (4-8 weeks after the EoT visit), and then enter phone follow-up directly. Follow-up visits will be performed for the evaluation of survival status, safety parameters, QoL/pain and administration of other anti-tumor treatment until death or end of the study, whichever comes first.

Anti-tumor therapy after termination of study treatment is at the discretion of the Investigator. However, the OFF regimen (O=Oxaliplatin; F=Fluorouracil; F=Leucovorin Calcium (Folinic Acid)) is recommended.

The cut-off date for the main analysis is 12 months after the last subject is randomized or the last subject alive has been followed up for at least 12 months, whatever applies first. Subjects being still under treatment with study medication at this cut-off date will enter the extension phase of this trial. These subjects are followed up until 28 days after the last administration of study medication.

1.4 Selection of study population

Inclusion Criteria

Potential subjects are required to meet all of the following criteria for enrollment into the study and subsequent randomization:
1. Age≥18 years
2. Written informed consent
3. Histologically or cytologically confirmed adenocarcinoma of the pancreas
4. Metastatic or locally advanced disease that is considered unresectable
5. Measurable/assessable disease according to RECIST v.1.1
6. Documented disease progression on first line FOLFIRINOX
7. Negative pregnancy test
8. Willingness to perform double-barrier contraception during study and for 4 weeks after last treatment
9. ECOG performance status 0 or 1

Exclusion Criteria

Patients who meet one or more of the following criteria cannot be considered eligible to participate in the study:
1. Cardiovascular disease, New York Heart Association (NYHA) III or IV
2. History of severe supraventricular or ventricular arrhythmia
3. History of coagulation or bleeding disorder
4. History of acute myocardial infarction within 6 months before randomization
5. History of congestive heart failure
6. Acute or chronic inflammation (autoimmune or infectious)
7. Significant active/unstable non-malignant disease likely to interfere with study assessments
8. Laboratory tests (hematology, chemistry) outside specified limits:
   a) WBC≤3×10³/mm³
   b) ANC≤1.5×10³/mm³
   c) Platelets≤100.000/mm³
   d) Hb≤9.0 g/dl (≤5.6 mmol/l)
   e) PTT>1.5×ULN specified limits:
   f) Serum creatinine>2.0 mg/dl (>176.8 μmol/l)
   g) AST and/or ALT>2.5×ULN; for patients with significant liver metastasis AST and/or ALT>5×ULN
   h) Alkaline phosphatase>2.5×ULN
   i) Total bilirubin>2×ULN
   j) Albumin<2.5 g/dL
9. Clinically significant ascites
10. Immunotherapy<6 weeks prior to enrollment.
11. Any anti-tumor treatment (except FOLFIRINOX as the first-line therapy) for pancreatic adenocarcinoma before enrollment. Note: Subjects who have undergone surgical interventions for pancreatic adenocarcinoma will be eligible.
12. Any radiotherapy for pancreatic adenocarcinoma before enrollment except for treatment of bone metastases if target lesions are not included in the irradiated field
13. Major surgery<4 weeks prior to enrollment
14. Pregnant or nursing
15. Investigational medicinal product<4 weeks of enrollment
16. Documented HIV history
17. Active hepatitis B or hepatitis C
18. Known hypersensitivity to any component of the EndoTAG®-1 and/or gemcitabine formulations
19. History of malignancy other than pancreatic cancer<3 years prior to enrollment, except non-melanoma skin cancer or carcinoma in situ of the cervix treated locally
20. Vulnerable populations (e.g. subjects unable to understand and give voluntary informed consent)

2.5 Drug Administration

Arm A:

Treatment cycle 1: EndoTAG®-1 is given at a dose of 22 mg/m² as an intravenous infusion which should be started slowly and increased to a maximum of 1.5 ml/min (15 min at 0.5 ml/min, 15 min at 1.0 ml/min. and thereafter 1.5 ml/min.) on days 1, 4, 8, 11, 15, 18, 22, 25, 29, 32, 36, 39, 43 and 46 plus gemcitabine 1000 mg/m², 30 min. i.v. infusion on days 4, 11, 18, 25, 32, 39, and 46 of cycle 1 until any one of the following occurs: progressive disease or unacceptable toxicity or withdrawal of patient consent

Subsequent treatment cycles: EndoTAG®-1 on days 1, 4, 8, 11, 15, and 18 plus gemcitabine on days 4, 11, and 18 of all subsequent cycles, until any one of the following occurs: progressive disease or unacceptable toxicity or withdrawal of patient consent

Arm B:

Treatment cycle 1: Gemcitabine 1000 mg/m², 30 min. i.v. infusion on days 4, 11, 18, 25, 32, 39, and 46 of cycle 1 until any one of the following occurs: progressive disease or unacceptable toxicity or withdrawal of patient consent

Subsequent treatment cycles: Gemcitabine on days 4, 11, and 18 of all subsequent cycles, until any one of the following occurs: progressive disease or unacceptable toxicity or withdrawal of patient consent

2.6 Dose adjustment in the event of toxicities:

The doses and timing of treatment is modified based on toxicities experienced by the patient. Dose modification and retreatment are outlined below:

Dose Modifications for EndoTAG®-1:

Criteria for Dose Modifications
Grade 4 neutropenia lasting 7 or more days
Febrile neutropenia
Grade 4 thrombocytopenia
Grade 3 thrombocytopenia with significant bleeding or requiring transfusion
Grade≥3 stomatitis/vomiting/diarrhea
Other≥Grade 3 and 4 toxicities (Except Grade 3 fatigue/asthenia or transient arthralgia/myalgia for which no dose modification is required.)

If any of the above mentioned toxicity criteria are present, no study medication is administered at this visit. If the toxicity criteria are no longer fulfilled at the next scheduled visit, EndoTAG®-1 is administered at a reduced dose of 11 mg/m². If the subject tolerates treatment at the reduced dose (i.e. does not develop any of the above mentioned toxicities), EndoTAG®-1 dose should be re-escalated to 22 mg/m². If re-escalation is not tolerated by the subject, the dose is permanently reduced to 11 mg/m². The attempt for re-escalation of the EndoTAG-1 dose is made only once throughout the study.

Dose Modifications for Gemcitabine:

Dose Modifications for Hematologic Adverse Reactions

| Absolute granulocyte count | | Platelet count | |
|---|---|---|---|
| (×10⁶/L) | | (×10⁶/L) | % of full dose |
| ≥1000 | And | ≥100,000 | 100% |
| 500-999 | Or | 50,000-99,999 | 75% |
| <500 | Or | <50,000 | Hold |

Dose Modifications for Non-Hematologic Adverse Reactions

Permanently discontinue Gemcitabine for any of the following:
Unexplained dyspnea or other evidence of severe pulmonary toxicity
Severe hepatic toxicity
Hemolytic-uremic syndrome
Capillary leak syndrome
Posterior reversible encephalopathy syndrome

Withhold gemcitabine or reduce dose by 50% for other severe (Grade 3 or 4) non-hematological toxicity until resolved.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A method of treating refractory or resistant pancreatic cancer, wherein the method comprises administering to a subject in need thereof (a) a cationic liposomal formulation comprising one or more cationic lipids and a therapeutically effective amount of paclitaxel, and (b) a therapeutically effective amount of gemcitabine.

2. The method of claim 1, wherein the refractory or resistant pancreatic cancer is refractory or resistant to one or more antineoplastic agents comprising fluorouracil, bleomycin, bortezomib, carboplatin, cisplatin, cytarabine, docetaxel, doxorubicin, elmustin, erlotinib, etoposide, gemcitabine, idarubicin, imatinib, lomustine, methotrexate, mitomycin, mitoxantrone, oxaliplatin, paclitaxel, pemetrexed, sunitinib, topotecan, treosulfan, vemurafenib, vinblastine, vincristine, vindesine, or vinorelbine.

3-5. (canceled)

6. The method of claim 1, wherein the refractory or resistant pancreatic cancer is refractory or resistant to a growth factor inhibitor.

7. (canceled)

8. The method of claim 1, wherein the refractory or resistant pancreatic cancer is refractory or resistant to an antimitotic agent.

9. (canceled)

10. The method of claim 4, wherein the subject has previously been treated with an intravenous infusion of about 70 mg/m² to 100 mg/m² oxaliplatin followed by an intravenous infusion of about 300 mg/m² to 500 mg/m² leucovorin concomitantly with an intravenous infusion of about 90 mg/m² to 270 mg/m² irinotecan, followed by an intravenous bolus of about 300 mg/m² to 800 mg/m² fluorouracil and an intravenous infusion of about 1200 mg/m² to 3600 mg/m² fluorouracil.

11. (canceled)

12. The method of claim 2, wherein the subject has previously been treated with one or more antineoplastic agent without administering a pulsed dose.

13. The method of claim 12, wherein the subject has previously been treated with an intravenous infusion of about 70 mg/m² to 100 mg/m² oxaliplatin followed by an intravenous infusion of about 300 mg/m² to 500 mg/m² leucovorin concomitantly with an intravenous infusion of about 90 mg/m² to 180 mg/m² irinotecan, followed by an intravenous infusion of about 1200 mg/m² to 3600 mg/m² fluorouracil.

14. (canceled)

15. The method of claim 1, wherein the method comprises administering about 1 mg/m² to about 60 mg/m² paclitaxel in the cationic liposomal formulation and about 300 mg/m² to about 1500 mg/m² gemcitabine to the subject.

16. (canceled)

17. The method of claim 1, wherein the cationic liposomal formulation is administered twice weekly, and gemcitabine is administered once weekly.

18. The method of claim 15, wherein the cationic liposomal formulation is administered on days 1, 4, 8, 11, 15, 18, 22, 25, 29, 32, 36, 39, 43, and 46 and gemcitabine is administered on days 4, 11, 18, 25, 32, 39, and 46 of a first treatment cycle of seven weeks.

19. The method of claim 18, wherein the first treatment cycle is followed by one or more subsequent treatment cycles, the cationic liposomal formulation is administered on days 1, 4, 8, 11, 15, and 18 and gemcitabine is administered on days 4, 11, and 18 of a subsequent treatment cycle of three weeks, and a dosing interval between a first treatment cycle and a subsequent treatment cycle or between two subsequent treatment cycles is one week.

20. The method of claim 1, wherein the cationic liposomal formulation is administered to the subject at a rate of 0.5 mL/min for first 15 minutes, followed by a rate of 1.0 mL/min for second 15 minutes, and followed by a rate of 1.5 mL/min after 30 minutes.

21. A method of treating refractory or resistant pancreatic cancer, wherein the method comprises administering to a subject in need thereof a cationic liposomal formulation comprising one or more cationic lipids and a therapeutically effective amount of paclitaxel.

22. The method of claim 21, wherein the refractory or resistant pancreatic cancer is refractory or resistant to one or more antineoplastic agents comprising fluorouracil, bleomycin, bortezomib, carboplatin, cisplatin, cytarabine, docetaxel, doxorubicin, elmustin, erlotinib, etoposide, gemcitabine, idarubicin, imatinib, lomustine, methotrexate, mitomycin, mitoxantrone, oxaliplatin, paclitaxel, pemetrexed, sunitinib, topotecan, treosulfan, vemurafenib, vinblastine, vincristine, vindesine, or vinorelbine.

23-27. (canceled)

28. The method of claim 21, wherein the refractory or resistant pancreatic cancer is refractory or resistant to an antimitotic agent.

29. (canceled)

30. The method of claim 1, wherein the cationic liposomal formulation comprises a cationic lipid from about 30 mole % to about 99.9 mole %, paclitaxel in an amount of at least 0.1 mole % and a neutral or an anionic lipid in an amount of 30 mole % to 55 mole %, and the cationic liposomal formulation has a positive zeta potential in about 0.05 M KCl solution at about pH 7.5 at room temperature.

31-39. (canceled)

40. The method of claim 1, wherein the cationic liposomal formulation and the gemcitabine are administered simultaneously but separately.

41. The method of claim 1, wherein the cationic liposomal formulation and the gemcitabine are administered sequentially.

42. A method of inhibiting the growth of multidrug resistant (MDR) pancreatic cells comprising administering to MDR pancreatic cells a cationic liposomal formulation comprising one or more cationic lipids and a therapeutically effective amount of paclitaxel.

43. The method of claim 42, wherein the method further comprises administering a therapeutically effective amount of gemcitabine.

44. The method of claim 43, wherein the cationic liposomal formulation and the therapeutically effective amount of gemcitabine are administered simultaneously, but separately, or are administered sequentially.

45. The method of claim 1, wherein the method is preceded by a neoadjuvant therapy.

46. (canceled)

47. (canceled)
